(19) United States
(12) Patent Application Publication
(10) Pub. No.: US 2015/0301035 A1
(43) Pub. Date: Oct. 22, 2015

(54) CALIBRATION STRIP FOR AN IMMUNOBLOT

(71) Applicant: Euroimmun Medizinische Labordiagnostika AG, Lubeck (DE)

(72) Inventors: Wolfgang Meyer, Pansdorf (DE); Thomas Scheper, Berkenthin (DE)

(21) Appl. No.: 14/346,262

(22) PCT Filed: Sep. 19, 2012
(86) PCT No.: PCT/EP2012/068381

§ 371 Date: Mar. 20, 2014

(30) Foreign Application Priority Data
Sep. 24, 2011 (DE) 202011 106 093.8

(51) Int. Cl. G01N 33/543

(52) U.S. Cl. CPC G01N 33/54386

(57) ABSTRACT

An apparatus for detecting antibodies in a patient sample includes at least one incubation channel for accommodating a patient strip and for incubating the patient strip with a patient sample, a conjugate and a substrate and a control mechanism for visual inspection of incubation quality. A separate calibration strip has at least two control zones which are implemented in such a way that, owing to incubation with a reference sample, a conjugate and a substrate, a control band becomes visible in each case in the control zones in such a way that the color intensities of the control bands differ. An evaluation unit generates a calibration curve taking the different color intensities into account and ascertains, on the basis of the calibration curve, a quality value for an antibody-pathogen protein reaction which took place on the patient strip and information about antibody concentration in the patient sample examined.

The invention relates to a method and an apparatus for detecting antibodies in a patient sample, having at least one incubation channel into which it is possible to insert an immunoblot strip and to incubate said strip with a patient sample, a conjugate and a substrate. The apparatus additionally has control means for visual inspection of incubation quality.

In the field of medical laboratory diagnostics, various assay systems are known, with which the presence of specific antibodies in patient samples is tested. Since antibodies are formed in the body of a patient after a viral or bacterial infectious disease, using such assays makes it possible to diagnose acute diseases or else diseases which the patient has already experienced. Similarly, it is also possible to diagnose autoimmune diseases or allergies when correspondingly suitable antigens are selected.

Typically, antibodies are detected by means of so-called stepwise diagnostics, in which firstly sensitive screening and then specific confirmation are carried out. In this connection, in routine serology, ELISAs (enzyme-linked immunosorbent assay) are often used for the screening, whereas immunoblot strips, more particularly Western blot strips, dot blot strips or line blot strips, are primarily used as a confirmation assay.

By detecting specific patient antibodies and the particular immunoglobulin classes to which they belong, it is possible to draw conclusions both about a bacterial or viral infection which has already occurred or an acutely existing bacterial or viral infection, and about the progress of an infection over time. For instance, antibodies of immunoglobulin class IgM are so-called early-phase antibodies, whereas antibodies of immunoglobulin class IgG are typically late-phase antibodies.

To ensure that the particular assays have been carried out correctly and thus show the right results, immunoblots having so-called function or reaction control bands are known, by means of which the addition of either serum or conjugate can be checked. Here, the conjugate contains an antibody which is coupled to the specific antibody in the patient serum and which generally originates from another animal species. The corresponding control bands always appear when a conjugate of any Ig class has been added.

However, by contrast, it is not possible with these assay systems to receive assurance as to whether the conjugate of a specific Ig class that is required for the assay was used. For instance, in the case of an assay designed in this way, a band would always appear when the test strip has been incubated with a conjugate; specifically, this would occur irrespective of whether IgM conjugate, IgG conjugate or IgA conjugate was used. In this way, it might be possible to overlook, for example, a Borrelia disease in the early phase if an IgM test strip was wrongly incubated with IgG conjugate, since although the control band would appear, the actual assay would deliver a negative result. Thus, in this case, a therapy would cease in the early phase of the disease and the infection could switch unnoticed into the late phase.

For this reason, it is desirable if, on the basis of a test strip, it could be identified whether the serum has been added and whether the functionality of the added conjugate is correct.

In this connection, DE 100 00 322 A1 discloses an immunoblot strip having at least two different control zones. The described test strips have, firstly, a serum control zone, which indicates by means of a corresponding control band that the test strip has been incubated with patient serum, and also, secondly, at least one conjugate control zone, which indicates that the test strip has been incubated with a labeled anti-patient immunoglobulin antibody from another animal species. By providing multiple different conjugate control zones, it is possible to determine with a corresponding test strip whether the conjugate provided for the test has actually been used. In addition, it is optionally provided to have on the test strip a cut-off sensor zone which makes it possible to check that the correct incubation time has been complied with.

As an alternative to the cut-off sensor zone on the test strip, it is further known to provide a separate predeveloped calibration strip in order to compare the color intensity of the cut-off control on the patient strip with the corresponding sensor zone of the calibration strip (see the instruction sheet for ORG 710 ANA-9-Line [710-0803-02-d] from Orgentec Diagnostika GmbH, March 2008). The cut-off control of the patient strip is “applied” to the corresponding band on the calibration strip and the color intensities are compared. In this way, the intention is to check whether the test has been carried out under reliable conditions.

A problem of the solutions known from the prior art is that the experimental conditions actually prevailing in the laboratory are often not satisfactorily taken into account when evaluating the assays. For instance, a test strip which has already been preincubated does not allow any conclusions to be drawn about the intensity in which the individual bands would appear under different laboratory conditions. Similarly problematic is the fact that while the frequently used positive and/or negative controls allow statements to be made as to whether the incubation with patient serum and/or with the correct conjugate occurred, quantitative statements with regard to the assay results, especially while taking into account specific properties of the bands appearing on the particular test strip, are by contrast not possible. In addition, the known assay systems either do not check the compliance with the correct incubation time while taking into account the actual laboratory and assay conditions, or the corresponding evaluation is associated with considerable effort, since each test strip needs to be individually checked. In this connection, assay kits, for example, are known, in which a cut-off zone is provided on each test strip and an incubation has to be stopped once a cut-off band appears on the test strip. Performing a test in this way appears to be disadvantageous, particularly with respect to the development in the field of laboratory technology toward an increasingly greater degree of automation, since a test strip has to be observed over the entire incubation.

Proceeding from the known assay systems and also the associated problems, it is an object of the invention to specify an assay system for evaluating immunoblot strips, which by means of comparatively simple means allows, firstly, checking of experimental parameters, more particularly the addition of patient serum and/or of the correct conjugate, and, secondly, calibration of the test results while taking into account the actually prevailing experimental conditions. Furthermore, the assay system to be specified should make it possible or be at least appropriately easily adaptable such that monitoring of the correct incubation time is realized. Here, a cut-off sensor zone should be particularly effectively arranged within the assay system. In addition, additional control zones, for example in the form of positive or negative control, should be easily integratable into the assay system to be specified.

The object underlying the invention is achieved by an apparatus for determining antibodies in a patient sample as claimed in claim 1 and also by a method as claimed in claim 8. Advantageous embodiments of the invention are subject matter of the dependent claims and are more particularly elucidated in the following description with partial reference to the figures.

According to the invention, an apparatus for detecting antibodies in a patient sample, having at least one incubation channel into which it is possible to insert an immunoblot strip and to incubate said strip with a patient sample, a conjugate and a substrate, and having a control means for visual inspection of incubation quality, has been further developed such that a separate calibration strip having at least two control zones is provided as control means, which is insertable into a further incubation channel and incubatable with a known reference sample, the conjugate and the substrate, and so, after incubation has been carried out, it is possible to ascertain a quality value for an antibody-pathogen protein reaction on the basis of a comparison of a color intensity of the control bands appearing in the at least two control zones of the calibration strip with a color intensity of at least one band of the at least one incubated incubation strip. A quality value is understood with regard to the apparatus according to the invention to mean that it is possible to ascertain the quality of the assay carried out with respect to at least one criterion.

The technical solution according to the invention provides an assay system which makes it easily possible to check the quality of an incubation while taking into account the laboratory conditions actually prevailing during the incubation. Here, it is conceivable to compare the intensity of the control bands appearing on the calibration strip with at least one band appearing on a patient strip and/or to create a calibration curve taking the at least two control bands on the calibration strip into account, which curve is ultimately consulted when evaluating the patient strips.

In a specific embodiment of the invention, the at least two control bands on the calibration strip appear at varying intensity at the same antibody concentration in the patient sample. The intensities of the control bands are captured and a calibration curve is created from these values. On the basis of said calibration curve, it is possible, firstly, to make statements about the quality of the incubation carried out and, secondly, to at least semiquantitatively evaluate the bands appearing on the patient strips. To further improve the calibration, preferably more than two, in particular five, control bands are provided on the calibration strip in order to create a calibration curve. Once a calibration curve has been created after completion of an assay, it is checked whether the calibration curve is within the range of values specified on the batch certificate belonging to the particular assay.

A specific further development of the invention further envisages that a control zone for a negative control is provided on the calibration strip. In the control zone of the negative control, typically no band appears, since it is either coated only very thinly or reacts to antibodies which are normally not to be expected in the patient sample. If a band appears in the control zone of the negative control, it should be checked in any case whether the assay was carried out correctly, since it is at least highly unlikely that the reference sample examined reacts positively with regard to antigen located in said control zone.

In another specific embodiment of the invention, a control zone for a positive control is provided on the calibration strip. In said control zone, a band appears when the calibration strip has been incubated with the patient sample. On the basis of the positive control, it is checked whether the calibration strip and thus the patient strips incubated in the same assay run have been incubated with the patient sample.

In a further preferred design of the invention, it is conceivable to provide a cut-off control zone on the calibration strip. With the aid of the cut-off control, it is checked whether the planned period for incubation of the test strips has been complied with. It is always important when carrying out the assays that the calibration strip is incubated under the same conditions as the patient strips processed together therewith.

A further embodiment envisages that a calibration strip implemented according to the invention is provided with a control zone on which a band appears once the calibration strip has been incubated with conjugate. In this connection, the calibration strip advantageously has at least two conjugate control zones which react to antibodies of the patient sample of different immunoglobulin classes. Preferably, at least one IgG conjugate control and one IgM conjugate control are provided. A very specific further development of the invention furthermore envisages that an IgA conjugate control zone is also provided in addition to an IgG conjugate control zone and an IgM conjugate control zone.

The above-described conjugate control zones ensure that, firstly, it is checked whether the calibration strip and also the at least one patient strip incubated together therewith has been incubated with a conjugate, and, secondly, it is furthermore also possible to check whether the correct or required conjugate for this situation has been used.

The use of the conjugate of the prescribed immunoglobulin class is of importance in this respect because so-called early-phase antibodies (IgM) and late-phase antibodies (IgG) are formed depending on the particular disease. A patient sample containing early-phase antibodies, but no late-phase antibodies, for example against Borrelia, normally shows a positive IgM band and a negative IgG band. If, by mistake, an IgM patient strip should have been incubated with an IgG conjugate, this could lead to a false assay result, it being possible for in particular false-negative assay results, for example an undetected early-phase infection, to have serious consequences for the patient. For this reason, the use of both patient strips and calibration strips having separate zones for the detection of antibodies of different immunoglobulin classes is advantageous.

In this connection, it is likewise conceivable that not only or at least not only is the use of the correct conjugate checked, but also the presence of the antibody class to be examined. By means of an assay performance expanded in this manner, the patient sample can be examined in terms of whether antibody deficiency syndrome is present in the patient. An assay result can thus be secured with a relatively high degree of accuracy.

The binding of antigen to antibody is preferably visualized by means of labeled antibodies against the primary antibody from another animal species. Preferably, enzyme-conjugated rabbit or goat antibodies are used here.

Alternatively, a specific further development of the invention envisages that the assay kit is implemented in such a way that, after the incubation with the patient sample, the test strip is not incubated with a secondary antibody from another animal species, but directly with an anti-human IgG, and is then washed.

Since the assay system according to the invention involves the patient strips being incubated together with at least one calibration strip, the strips therefore being incubated under the same experimental conditions, it is sufficient merely to provide on the calibration strip appropriate control zones for a positive control, negative control, conjugate control and/or cut-off control and to check the calibration strip after incubation has been carried out. It is possible to dispense with the individual additional control zones otherwise often provided on the patient strips, and so more space for further antigens is present on said strips.

The invention further provides a method for detecting antibodies in a patient sample, in which a patient strip is firstly inserted into an incubation channel and incubated with a patient sample, a conjugate and a substrate and in which incubation quality is subsequently captured by visual inspection of a control means. The method according to the invention is notable for the fact that, as control means, a separate calibration strip having at least two control zones is inserted into a further incubation channel and incubated with a reference sample, a conjugate and a substrate, and that, after incubation has been carried out, a color intensity of control bands appearing in the at least two control zones of the calibration strip is compared with a color intensity of at least one band of the at least one incubated patient strip and, on the basis of the comparison, a quality value is ascertained for an antibody-pathogen protein reaction which took place on the patient strip.

The invention will be more particularly elucidated below on the basis of exemplary embodiments with reference to the figures, without restricting the general idea of the invention. In this connection:

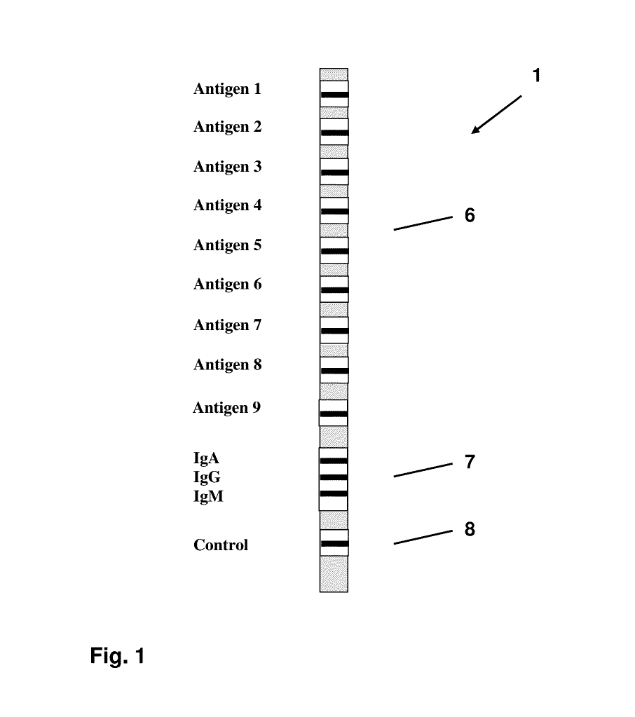

FIG. 1 shows: a conventional Western blot strip,

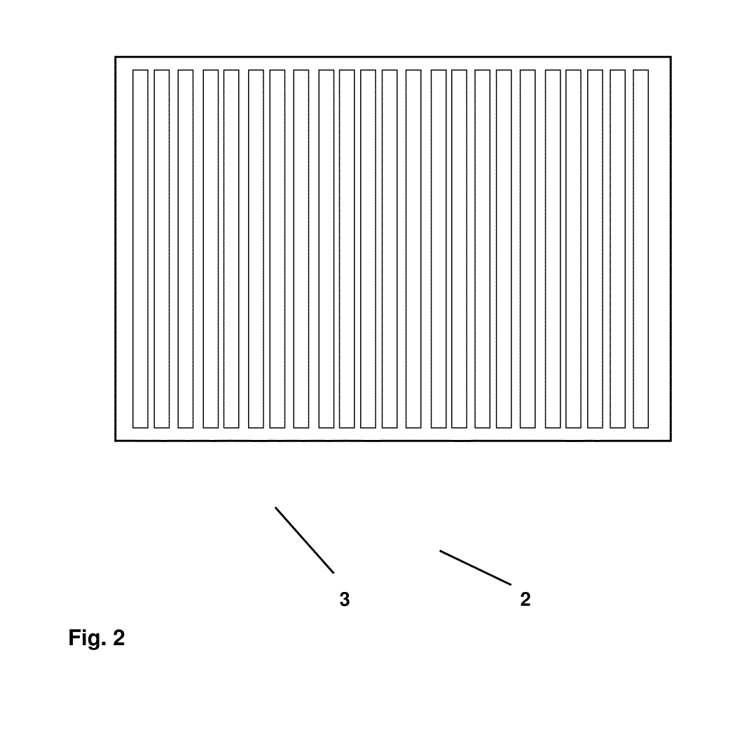

FIG. 2 shows: an incubation bath,

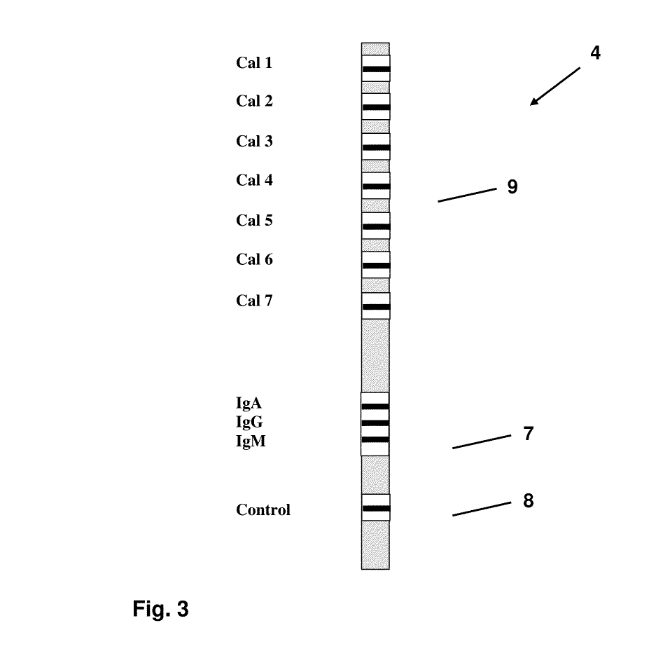

FIG. 3 shows: a calibration strip, and

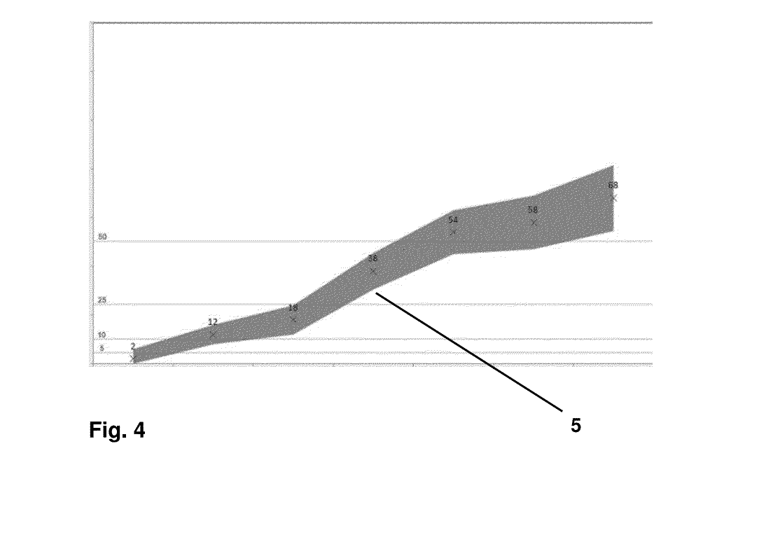

FIG. 4 shows: a graph with the calibration curve.

FIG. 1 shows a Western blot test strip 1 for examining a patient sample for a possible Borrelia infection. For the examination, the Western blot strip 1 is firstly inserted into an incubation channel 3 and incubated with a patient serum. For the incubation, an incubation bath 2 is typically used, as shown in FIG. 2. The incubation bath 2 has a plurality of incubation channels 3, making it possible to simultaneously incubate a corresponding number of test strips.

The test strip 1 or the test strips are incubated with an enzyme-coupled conjugate and also a substrate. Alternatively, it is conceivable to configure the assay in such a way that an anti-human IgG is used instead of the conjugate. Hereinafter, the performance of the assay is described merely by way of example using a labeled secondary antibody from another animal species, and this is therefore not intended to restrict the subject matter of the invention.

If the serum comprises antibodies based on a Borrelia infection, they bind to the pathogen proteins immobilized on the test strip. With the aid of the conjugate, the bound antibody is then detected by a second antibody from another animal species (rabbit, goat) that is labeled with an enzyme. Said detection is carried out by adding the substrate, which appropriately reacts with the enzyme of the conjugate. The assay result ultimately becomes apparent as a band pattern on the Western blot strip, which indicates the presence of a Borrelia infection. For this purpose, nine different antigens which bind to antibodies specific for a Borrelia infection are immobilized in the test zone 6 on the patient strip 1 depicted in FIG. 1.

The performance of the above-described assay, more particularly the sequence of the incubation steps, encompasses a multiplicity of possible sources of error. Therefore, especially in routine serology, there is the need to monitor the quality of the individual incubation steps. For this reason, additional control zones are provided on the Western blot strip depicted in FIG. 1. The control zones provided are a conjugate control zone 7 in which a band becomes apparent if an incubation with a conjugate has occurred, and also a positive control 8 which indicates an incubation with patient serum. Thus, the patient strip 1 has, firstly in test zone 6, regions in which corresponding bands are shown when a pathogen-protein reaction occurs. Secondly, serum and conjugate control zones 7, 8 are provided. It is important here that the conjugate control zone 7 is divided in turn into two or possibly three different regions in which a band appears in each case if IgG conjugate or IgM conjugate or, in the presence of a third region, IgA conjugate has been incubated. The patient strip 1 depicted in FIG. 1 does not comprise further function or reaction control bands.

Using the test strip 1 depicted in FIG. 1, the diagnosis of a Borrelia infection is carried out conventionally, with typically a plurality of test strips 1 being inserted into an incubation bath 2 comprising a multiplicity of parallel incubation channels 3, and incubated. In this way, a multiplicity of patient strips 1 can be incubated at the same time and thus different patient sera can be examined for a possible Borrelia infection. An incubation bath 2, often also referred to as a tray, having a plurality of incubation channels 3 is depicted in FIG. 2. The incubation channels 3 are preferably designed in such a way that, firstly, the strips 1 have sufficient space in the incubation channels and can be well flooded by the media used for the incubation and also, secondly, unnecessarily large amounts of medium need not be used for the incubation.

In FIG. 3, a calibration strip 4, as used in an assay system implemented according to the invention, is depicted. The calibration strip 4 has a calibration zone 9, a conjugate control zone 7 and also a positive control 8. The calibration strip 4 is provided as a separate strip which is incubated together with the patient strip(s) 1 and thus under the same experimental conditions. For the incubation, both a calibration strip 4 and the patient strips 1 are inserted into separate incubation channels 3 of an incubation bath 2, with the patient strips 1 being incubated with patient serum, conjugate and substrate and the calibration strip 4 being incubated with a reference sample, conjugate and substrate.

In its calibration zone, the calibration strip 4 has seven regions in which calibration bands differing from one another in their intensity appear in each case following correct incubation with the reference sample under the actually prevailing laboratory conditions. Moreover, a conjugate control zone 7 having three different regions is provided, with a band appearing in each case in these regions depending on the conjugate used, i.e., depending on whether IgG conjugate, IgM conjugate or IgA conjugate has been used. Providing a conjugate control 7 having multiple regions thus makes it possible not only to check whether a conjugate has been incubated, but also to additionally check whether the conjugate of the conjugate class required at the time has been used.

In addition, the calibration strip 4 has a positive control zone 8 in which a band appears once the strip has been incubated with patient serum.

To complement the positive control 8, it is conceivable to provide a negative control zone which has been coated thinly with an antigen to such an extent that no band appears in said zone under normal conditions. If a band becomes apparent in this region after incubation has been carried out, the assay should be repeated in any case to safely rule out a misdiagnosis.

Besides the above-described control zones, it is further possible to provide on the calibration strip 4 a cut-off control zone in which a cut-off control band appears when the incubation period has been sufficiently long in order to ensure reliable assay results.

The calibration strip 4 depicted in FIG. 3 thus has, firstly, a calibration zone 9 which is divided into seven regions and in which bands of varying intensity arise once an incubation has been carried out correctly, and also, secondly, regions for a conjugate control 7 and a positive control.

What is important here is the provision of a calibration zone 9 in which control bands of varying intensity appear after correct incubation with a known reference sample. On the basis of these seven control bands, a calibration curve 5 is ultimately created, as can be seen in FIG. 4. After creation of the calibration curve 5, it is firstly checked whether the calibration curve is situated within the permissible limits for the corresponding batch of an assay kit. If this is the case, this demonstrates correct performance of the assay. In addition, the bands appearing in the test zone 6 on the patient strip(s) 1 simultaneously incubated with the calibration strip 4 are compared with the values of the calibration curve 5, i.e., with the intensities of the seven bands in the calibration zone 9 on the calibration strip 4.

On the basis of a comparison of the bands appearing on the calibration strip 4 with the corresponding bands of the patient strip 1, it is possible, firstly, to monitor the quality of the incubation carried out and, secondly, to perform an at least semiquantitative evaluation of the incubated patient strip 1. To this end, the bands depicted in the test zone 6 on the patient strip 1 with regard to the antigens 1 to 7 are compared with the bands in the calibration zone 9 of the calibration strip 4. On the basis of a comparison of the intensities of the bands, the particular titer of the patient sample examined can be inferred.

Preferably, both the creation of the calibration curve 5 and the capture of the bands appearing on the patient strip 1 and/or the comparison of the patient bands with the calibration bands take place automatically. After incubation has been carried out, the test strips 1 are placed or stuck on a suitable base and the surfaces of the calibration strip 4 and of the patient strips 1 are photographed or scanned and transferred to an electronic data-processing unit running preferably a specific laboratory software.

As an addition, it is conceivable for an evaluation software to subtract, from the input signal representing the recorded color intensity, a proportion of the signal caused by the recording of the background.

The central data-processing unit evaluates the intensities of the individual bands, making it possible for the calibration curve 5 and the corresponding assay results to be outputted via an output unit, preferably a monitor or a printer.

On the basis of a comparison of the intensities of the seven calibration bands in the calibration zone 9 of the calibration strip 4, which have varying intensities, with the bands shown in the test zones 6 on the patient strip(s) 1, it is possible to obtain information about the antibody concentration in the patient sample examined and to thus obtain an at least semiquantified assay result. It is important here that the incubation of the calibration strip 4 took place under the same experimental conditions as those of the patient strip(s) 1.

### LIST OF REFERENCE SIGNS

1 Patient strip
2 Incubation bath
3 Incubation channel
4 Calibration strip
5 Calibration curve
6 Test zone
7 Conjugate control zone
8 Positive control
9 Calibration zone

1-8. (canceled)

9. An apparatus for detecting antibodies in a patient sample, comprising:
at least one incubation channel (3) for accommodating a patient strip (1) and for incubating the patient strip (1) with a patient sample, a conjugate and a substrate;
a control means for visual inspection of incubation quality;
an evaluation unit and
a separate calibration strip (4) as the control means, the calibration strip (4) having at least two control zones which are implemented in such a way that, owing to incubation with a reference sample, a conjugate and a substrate, a control band becomes visible in each case in the control zones in such a way that the color intensities of the control bands differ, and
wherein the evaluation unit is configured to generate a calibration curve taking different color intensities into account and, wherein said evaluation unit is further configured to ascertain, on the basis of the calibration curve, a quality value for an antibody-pathogen protein reaction which took place on the patient strip (1) and information about antibody concentration in the patient sample examined.

10. The apparatus according to claim 9, wherein the control bands in the at least two control zones on the calibration strip (4) appear in varying color intensity at the same antibody concentration in the patient sample.

11. The apparatus according to claim 9, wherein the control bands in the at least two control zones on the calibration strip (4) each react specifically to antibodies provided in a reference sample.

12. The apparatus according to claim 9, wherein the calibration strip (4) has a positive control zone which appears as a band once the calibration strip (4) has been incubated with patient serum, conjugate and substrate.

13. The apparatus according to claim 9, wherein the calibration strip (4) has a negative control zone which is coated more thinly with an antigen than in a positive control zone or in which the antigen is directed to an antibody not suspected in the patient serum.

14. The apparatus according to claim 9, wherein the calibration strip (4) has at least one conjugate control zone in which a band appears once the calibration strip (4) has been incubated with the conjugate.

15. The apparatus according to claim 9, wherein the calibration strip (4) has an IgM conjugate control zone, an IgG conjugate control zone and an IgA conjugate control zone in which a band appears in each case once the calibration strip has been incubated with an IgM conjugate, IgG conjugate and/or IgA conjugate.

16. A method for detecting antibodies in a patient sample, comprising the steps of:
inserting a patient strip (1) into an incubation channel (3);
incubating said patient strip (1) with a patient sample, a conjugate and a substrate;
capturing incubation quality by visual inspection of a control means, wherein as control means, a separate calibration strip (4) having at least two control zones is inserted into a further incubation channel (3) and incubated with a reference sample, a conjugate and a substrate under the same experimental conditions as the patient strip (1), a control band becoming visible in each case in the control zones after the calibration strip (4) has been incubated, the color intensities of the bands differing;
generating a calibration curve (5) taking the different color intensities into account, wherein the curve is compared with a color intensity of at least one band of the at least one incubated patient strip (1); and
on the basis of the comparison, ascertaining a quality value for an antibody-pathogen protein reaction which took place on the patient strip (1) and ascertaining information about antibody concentration in the patient sample examined.
